(19) United States
(12) Patent Application Publication
(10) Pub. No.: US 2024/0335125 A1
(43) Pub. Date: Oct. 10, 2024

(54) PHYSIOLOGICAL INFORMATION MEASUREMENT DEVICE, PHYSIOLOGICAL INFORMATION MEASUREMENT METHOD, AND NON-TRANSITORY COMPUTER-READABLE STORAGE MEDIUM

(71) Applicant: NIHON KOHDEN CORPORATION, Tokyo (JP)

(72) Inventor: Shogo Totsuka, Tokorozawa-shi (JP)

(73) Assignee: NIHON KOHDEN CORPORATION, Tokyo (JP)

(21) Appl. No.: 18/620,021

(22) Filed: Mar. 28, 2024

(30) Foreign Application Priority Data
Apr. 7, 2023 (JP) 2023-062854

(51) Int. Cl. A61B 5/0225; A61B 5/00; A61B 5/021; A61B 5/022; A61B 5/352

(52) U.S. Cl. CPC A61B 5/0225; A61B 5/02108; A61B 5/02233; A61B 5/352; A61B 5/7203

(57) ABSTRACT

A physiological information measurement device includes a pressure controller configured to control an applied pressure of a cuff to a predetermined site of a subject, a pulse detector configured to detect pulses from a pressure received by the cuff from the predetermined site, a physiological information detector configured to detect physiological information on the subject, a noise classification unit configured to classify whether detection data detected by the pulse detector is noise based on the physiological information, and a blood pressure calculator configured to calculate a blood pressure of the subject based on a plurality of the pulses that are classified as not noise by the noise classification unit.

### CROSS-REFERENCE TO RELATED APPLICATIONS

The present application claims the benefit of priority of Japanese Patent Application No. 2023-062854, filed on Apr. 7, 2023, the content of which is incorporated herein by reference.

### TECHNICAL FIELD

The presently disclosed subject matter relates to a physiological information measurement device, a physiological information measurement method, and a non-transitory computer-readable storage medium.

### BACKGROUND ART

There has been an NIBP (non-invasive blood pressure) measurement device for measuring the blood pressure by wrapping a cuff around an upper arm or a lower leg of a subject, inflating the applied pressure of the cuff to be higher than the systolic blood pressure, and then deflating while detecting the pressure oscillation generated in the cuff due to pulses as an oscillation signal. In addition, there has been a non-invasive blood pressure measurement device that measures the blood pressure simultaneously with the inflation of the internal pressure of the cuff for the purpose of shortening the measurement time of the blood pressure (for example, see JPH08-322811A).

In the non-invasive blood pressure measurement device, during blood pressure measurement, noise may be generated due to a body motion or the like of the subject, and the noise may be erroneously detected as a pulse. In this case, since the trajectory (envelope) of the obtained oscillation signal changes, it may be difficult to calculate an accurate blood pressure. Therefore, there has been a non-invasive blood pressure measurement device that removes the noise by filtering the detected pressure oscillation using a filter parameter based on the heart rate of the subject (for example, see JP2016-174871A).

### SUMMARY

However, in the non-invasive blood pressure measurement device disclosed in JP2016-174871A, it may be not possible to completely remove the noise generated by the body motion or the like of the subject. In addition, the pressure oscillation generated in the cuff due to the pulse is also filtered, which lowers the blood pressure measurement accuracy. Furthermore, if the blood pressure measurement fails due to the noise and needs re-measurement, the burden on the subject increases because, for example, the measurement time is elongated.

An object of the presently disclosed subject matter is to provide a physiological information measurement device, a physiological information measurement method, and a program capable of maintaining the blood pressure measurement accuracy and reducing the burden on the subject by removing the noise caused by a body motion or the like of the subject during the measurement of the blood pressure.

According to a first aspect of the presently disclosed subject matter, there is provided a physiological information measurement device, including:
a pressure controller configured to control an applied pressure of a cuff to a predetermined site of a subject;
a pulse detector configured to detect pulses from a pressure received by the cuff from the predetermined site;
a physiological information detector configured to detect physiological information on the subject;
a noise classification unit configured to classify whether detection data detected by the pulse detector is noise based on the physiological information; and
a blood pressure calculator configured to calculate a blood pressure of the subject based on a plurality of the pulses that are classified as not noise by the noise classification unit.

According to a second aspect of the presently disclosed subject matter, there is provided a physiological information measurement method, including:
a pressure control step of controlling an applied pressure of a cuff to a predetermined site of a subject;
a pulse detection step of detecting pulses from a pressure received by the cuff from the predetermined site;
a physiological information detection step of detecting physiological information on the subject;
a noise classification step of classifying whether detection data detected in the pulse detection step is noise based on the physiological information; and
a blood pressure calculation step of calculating a blood pressure of the subject based on a plurality of the pulses that are classified as not noise in the noise classification step.

According to a third aspect of the presently disclosed subject matter, there is provided a non-transitory computer-readable storage medium storing a computer program executable by one or more processors provided in a computer, when being executed, the computer program causing the computer to execute:
a pressure control step of controlling an applied pressure of a cuff to a predetermined site of a subject;
a pulse detection step of detecting pulses from a pressure received by the cuff from the predetermined site;
a physiological information detection step of detecting physiological information on the subject;
a noise classification step of classifying whether detection data detected in the pulse detection step is noise based on the physiological information, and classifying the detection data if the detection data is noise; and
a blood pressure calculation step of calculating a blood pressure of the subject based on a plurality of the pulses that are classified as not noise in the noise classification step.

According to the presently disclosed subject matter, even if noise caused by the body motion or the like of the subject during the measurement of the blood pressure is erroneously detected as a pulse, it is possible to maintain the blood pressure measurement accuracy and reduce the burden on the subject by classifying the noise.

### BRIEF DESCRIPTION OF DRAWINGS

FIG. 1 is a block diagram illustrating a configuration of a physiological information measurement device according to embodiments of the presently disclosed subject matter.

FIG. 2 is a blood pressure measurement process flow according to a first embodiment of the presently disclosed subject matter.

FIG. 3 is a graph illustrating an example of a pulse detection result of a physiological information measurement device in the related art.

FIG. 4 is a graph illustrating an example of a pulse detection result of a physiological information measurement device according to the first embodiment of the presently disclosed subject matter.

FIG. 5 is a graph illustrating another example of the pulse detection result of the physiological information measurement device according to the first embodiment of the presently disclosed subject matter.

FIG. 6 is a graph illustrating an example of a pulse detection result of a physiological information measurement device according to a second embodiment of the presently disclosed subject matter.

### DESCRIPTION OF EMBODIMENTS

Hereinafter, embodiments of the presently disclosed subject matter will be described with reference to the drawings. Members having the same reference numerals as those already described in the description of the embodiment will not be described for convenience of description. Further, for convenience of description, dimensions of each member illustrated in the drawings may be different from actual dimensions of each member.

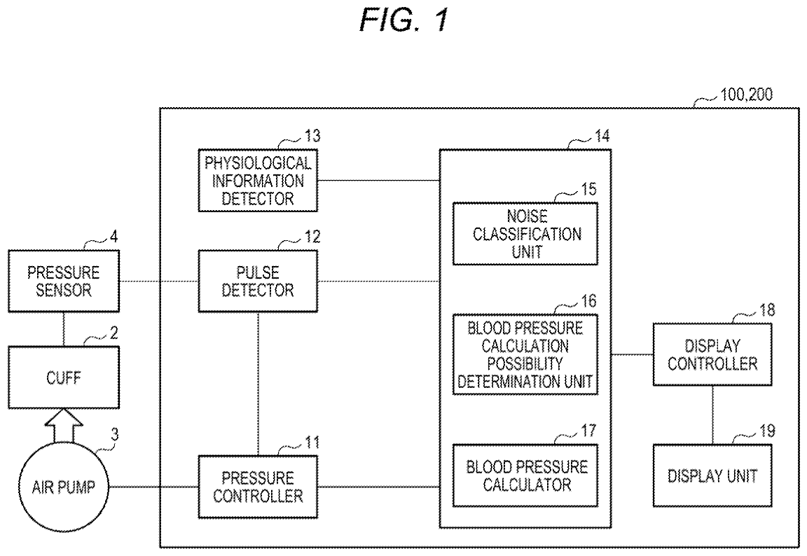

FIG. 1 is a block diagram illustrating a configuration of a physiological information measurement device 100 according to embodiments of the presently disclosed subject matter. The physiological information measurement device 100 may include a pressure controller 11, a pulse detector 12, a physiological information detector 13, a calculator 14, a display controller 18, and a display 19. The calculator 14 may include a noise classification unit 15, a blood pressure calculation possibility determination unit 16, and a blood pressure calculator 17. The physiological information measurement device 100 is configured to be connected to a cuff 2 that is to be wrapped around an upper arm or a lower limb of a subject, an air pump 3 that causes air to flow into the cuff 2, and a pressure sensor 4 that detects pressure oscillation generated in the cuff 2.

The pressure controller 11 adjusts the amount of air flowing into the cuff 2 by controlling the applied pressure of the air pump 3. Specifically, the pressure controller 11 performs at least once a measurement step of stepwise deflating the applied pressure from an initial value (for example, 100 mmHg) (for example, deflating the applied pressure by 5 mmHg at predetermined intervals). The pressure controller 11 may perform at least once a measurement step of inflating the applied pressure from an initial value (for example, 0 mmHg) at a predetermined ratio (for example, inflating the applied pressure by 25 mmHg per second). Further, the method of controlling the applied pressure may be changed for each measurement step, such as stepwise deflation in the measurement step of the first time and inflation at a predetermined ratio in the measurement step of the second time. The pressure controller 11 may control the magnitude of the applied pressure or the duration of application of the applied pressure based on the determination result of the blood pressure calculation possibility determination unit 16.

The pulse detector 12 detects the pulse of the subject (oscillation signal) from the pressure oscillation detected by the pressure sensor 4 at the time point when the applied pressure of the cuff 2 is deflated or inflated.

The physiological information detector 13 is, for example, an electrocardiograph, a pulse oxy meter, or the like, and detects physiological information on the subject. The physiological information may include at least one of the heart rate, the pulse rate, or the electrocardiogram.

The noise classification unit 15 determines whether the detection data (pulse) detected by the pulse detector 12 is noise based on the physiological information on the subject detected by the physiological information detector 13. The pulse is discarded if determined as noise. The noise determination method will be described in detail later.

The blood pressure calculation possibility determination unit 16 determines whether the blood pressure of the subject can be calculated based on a plurality of the pulses determined as not noise by the noise classification unit 15. Specifically, it is determined whether the systole and the diastole can be identified from the trajectory (envelope) based on the temporal transition of the plurality of pulses.

If the blood pressure calculation possibility determination unit 16 determines that the blood pressure of the subject can be calculated, the blood pressure calculator 17 identifies the systolic phase and the diastolic phase based on the plurality of pulses determined as detected correctly by the noise classification unit 15, and calculates the blood pressures (systolic blood pressure (SYS), diastolic blood pressure (DIA), and the mean arterial pressure (MAP)) of the subject.

The display controller 18 causes the display 19 to display various calculation results (measurement values) and waveforms including the blood pressures calculated by the blood pressure calculator 17. The display 19 is, for example, a liquid crystal display provided in the case of the physiological information measurement device 100. The display 19 may be a touch panel that receives an instruction related to an operation from a user. The display 19 may be configured to be attachable to and detachable from the physiological information measurement device 100, or may be a separate body connected to the physiological information measurement device 100 by a cable, Wifi, or the like.

### First Embodiment

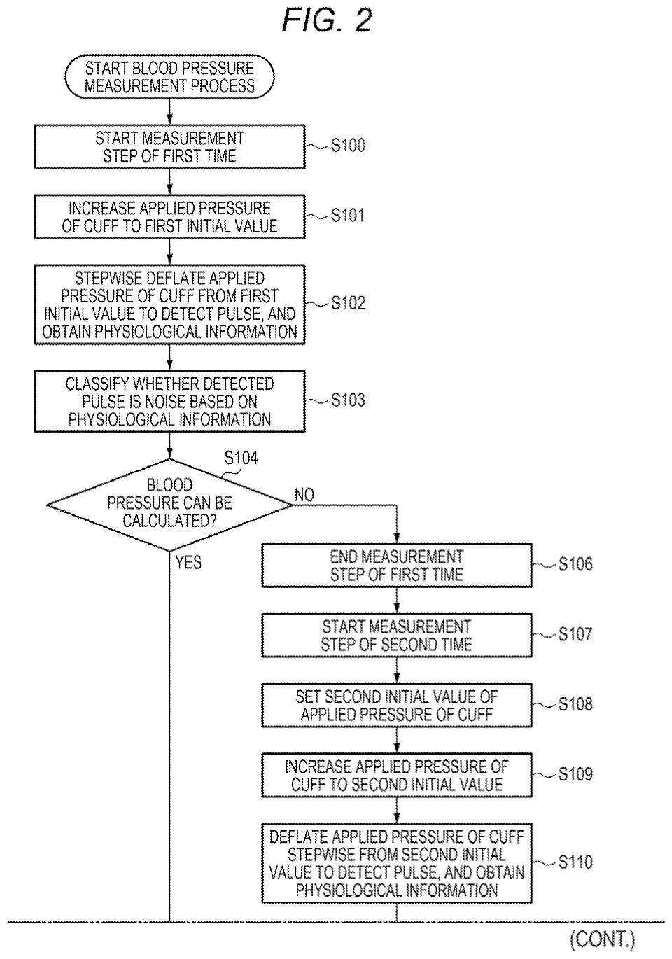

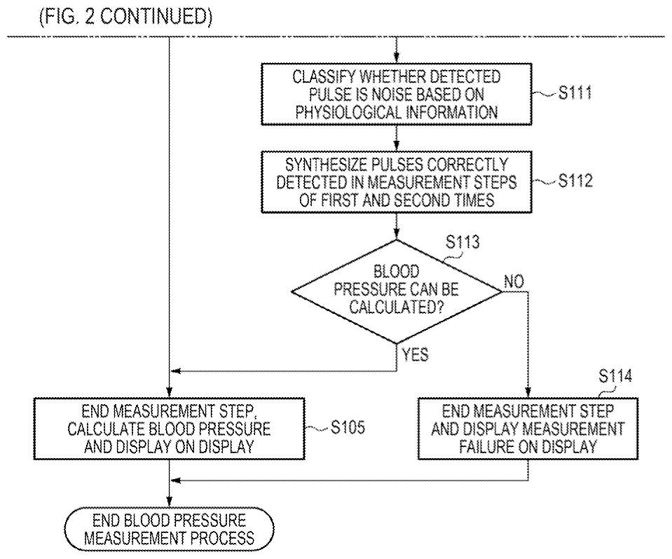

Hereinafter, the physiological information measurement device 100 according to the first embodiment will be described in detail with reference to FIGS. 2 to 5.

FIG. 2 illustrates a blood pressure measurement process flow according to the first embodiment 100 of the presently disclosed subject matter. The physiological information measurement device 100 according to the first embodiment is configured to stepwise deflate the applied pressure P of the cuff 2 in each measurement step.

When receiving a blood pressure measurement instruction from the user through the display 19 or the like, the physiological information measurement device 100 starts the blood pressure measurement process and starts the measurement step of the first time (S100).

Next, the pressure controller 11 controls the air pump 3 to inflate the applied pressure of the cuff 2 to a preset first initial value (S101). Thereafter, the pressure controller 11 controls the air pump 3 such that the applied pressure of the cuff 2 is stepwise deflated from the first initial value, and the pulse detector 12 detects the pulse of the subject from the pressure oscillation detected by the pressure sensor 4. Simultaneously with the detection of the pulse by the pulse detector 12, the physiological information detector 13 detects the physiological information on the subject (S102).

Next, the noise classification unit 15 classifies whether the pulse detected by the pulse detector 12 is noise based on the physiological information detected by the physiological information detector 13 (S103).

Next, the blood pressure calculation possibility determination unit 16 calculates the envelope and determines whether the blood pressure of the subject can be calculated based on the plurality of pulses classified as not noise by the noise classification unit 15 (S104). If it is determined that the blood pressure of the subject can be calculated (Yes in S104), the physiological information measurement device 100 ends the measurement step and cuts off the applied pressure of the cuff 2, the blood pressure calculator 17 calculates the blood pressure of the subject, and the display controller 18 causes the display 19 to display various calculation results including the calculated blood pressure (S105). If it is determined that the blood pressure of the subject cannot be calculated (No in S104), the physiological information measurement device 100 ends the measurement step of the first time (S106) and starts the measurement step of the second time (S107). At this time, the blood pressure calculation possibility determination unit 16 holds, rather than discards, the plurality of pulses determined as correctly detected in the measurement step of the first time.

After starting the measurement step of the second time (S107), the pressure controller 11 sets a second initial value of the applied pressure of the cuff 2 (S108). The second initial value may be the same value as the first initial value, or may be a value newly set based on the determination result of the noise classification unit 15.

Since subsequent steps S109 to S111 are the same as steps S101 to S103 described above, the description thereof will be omitted.

After classifying whether the pulse is detected correctly (S111), the blood pressure calculation possibility determination unit 16 synthesizes a plurality of pulses classified as not noise by the noise classification unit 15 in the measurement step of the first time and the measurement step of the second time (S112). The blood pressure calculation possibility determination unit 16 determines whether the blood pressure of the subject can be calculated based on the plurality of synthesized pulses (S113). If it is determined that the blood pressure of the subject can be calculated (Yes in S113), the physiological information measurement device 100 ends the measurement step and cuts off the applied pressure of the cuff 2, the blood pressure calculator 17 calculates the blood pressure of the subject, and the display controller 18 causes the display 19 to display various calculation results including the calculated blood pressure (S105). If it is determined that the blood pressure of the subject cannot be calculated (No in S113), the physiological information measurement device 100 ends the measurement step, and the display controller 18 causes the display 19 to display that the blood pressure cannot be measured and the measurement is ended (S114). Thereafter, the physiological information measurement device 100 ends the blood pressure measurement process.

In the process flow illustrated in FIG. 2, if the blood pressure cannot be calculated until the measurement step of the second time, the blood pressure measurement process is ended by causing the display 19 to display that the blood pressure cannot be measured and the measurement is ended, but the presently disclosed subject matter is not limited thereto. That is, the blood pressure measurement process may be ended by displaying on the display 19 that the blood pressure cannot be measured and the measurement is ended if the blood pressure cannot be calculated until measurement step of the Nth time (Nis an integer of 3 or more). In this case, the steps S106 to S112 are repeated in the measurement steps of the third and subsequent times.

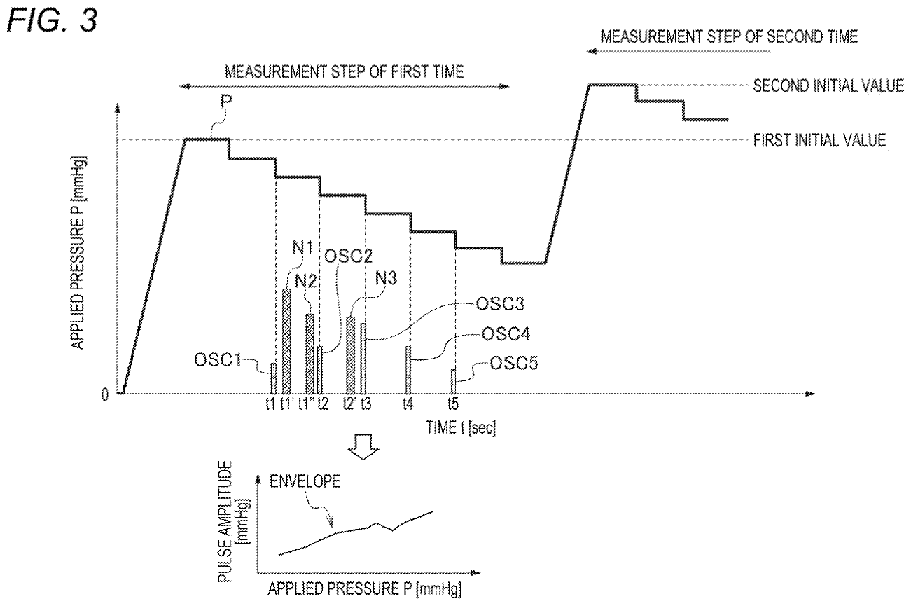

In order to describe a graph illustrating an example of the pulse detection result obtained using the physiological information measurement device 100 according to the first embodiment of the presently disclosed subject matter, for comparison, a graph illustrating an example of a pulse detection result obtained using the physiological information measurement device 100 in the related art will be described with reference to FIG. 3.

In FIG. 3, the horizontal axis represents the time t(s), and the vertical axis represents the applied pressure P (mmHg). In the measurement step of the first time, the applied pressure P of the cuff 2 is stepwise deflated from the first initial value. During this period, the pulse detector 12 detects the pulse of the subject from the pressure oscillation detected by the pressure sensor 4. In the example illustrated in FIG. 3, the pulse detector 12 detects the pulses OSC1 to OSC5 at the time points t1, t2, t3, t4, and t5. Further, the pulse detector 12 detects the noises N1 to N3 as pulses at the time points t1′, t1″, and t2′. If the envelope is generated from the pulses OSC1 to OSC5 and the noise N1 to N3, the pulses OSC1 and OSC2 are buried in the noise N1 to N3, and thus the waveform of the envelope increases monotonically as illustrated in the lower part of FIG. 3. Therefore, the peak value of the envelope cannot be identified, and the systole and diastole cannot be identified. Accordingly, the blood pressure calculation possibility determination unit 16 determines that the blood pressure cannot be calculated. At this time, the blood pressure calculation possibility determination unit 16 discards the pulses OSC1 to OSC5 and the noise N1 to N3.

In the measurement step of the second time, the initial value of the applied pressure P of the cuff 2 is set to the second initial value. At this time, since the waveform of the envelope generated in the measurement step of the first time monotonically increases and the peak value cannot be identified, the blood pressure calculation possibility determination unit 16 sets the second initial value to a value larger than the first initial value in the measurement step of the second time as illustrated in FIG. 3. Since the measurement step of the subsequent second time is the same or similar as the measurement step of the first time described above, the description thereof will be omitted.

As described above, in a physiological information measurement device 100 of the related art, the measurement accuracy of the blood pressure is reduced due to noise, and the burden on the subject is increased due to the increase in the applied pressure P and the increase in the measurement time in the measurement step of the second time.

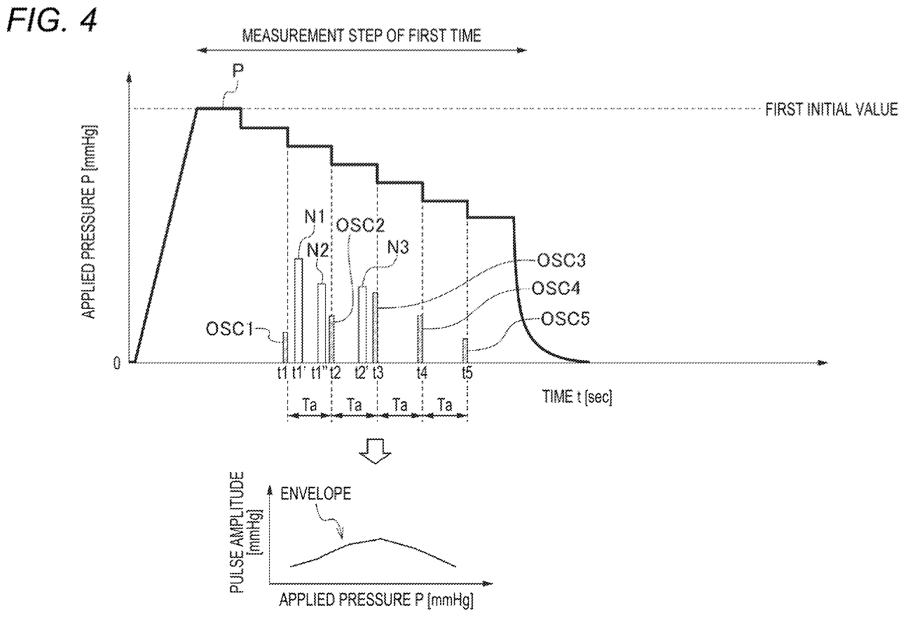

FIG. 4 is a graph illustrating an example of the pulse detection result of the physiological information measurement device 100 according to the first embodiment of the presently disclosed subject matter. The horizontal axis represents the time t(s), and the vertical axis represents the applied pressure P (mmHg).

In the measurement step of the first time, the applied pressure P of the cuff 2 is stepwise deflated from the first initial value. During this period, the pulse detector 12 detects the pulse of the subject from the pressure oscillation detected by the pressure sensor 4. In the example illustrated in FIG. 4, same or similar as the example illustrated in FIG. 3, the pulse detector 12 detects the pulses OSC1 to OSC5 at the time points t1, t2, t3, t4, and t5. Further, the pulse detector 12 detects the noises N1 to N3 as pulses at the time points t1′, t1″, and t2′. In the present embodiment, the noise classification unit 15 classifies whether the pulse detected by the pulse detector 12 is noise based on the physiological information detected by the physiological information detector 13. In the example illustrated in FIG. 4, the physiological information is the heart rate, and pulses detected at intervals shifted by a predetermined ratio (for example, +10%) or more from an average beat interval Ta (for example, 750 ms) calculated from the heart rate are classified as noise. As a result, the noises N1 to N3 are classified as noise and the pulses OSC1 to OSC5 are classified as not noise. If the envelope is generated from the pulses OSC1 to OSC5, since the waveform of the envelope is convex upward as illustrated in the lower part of FIG. 4, the peak value can be identified, and the systole and the diastole can be identified. Accordingly, the blood pressure calculation possibility determination unit 16 determines that the blood pressure can be calculated from the pulses OSC1 to OSC5, and the blood pressure calculator 17 calculates the blood pressure from the pulses OSC1 to OSC5.

The physiological information may be a pulse rate, and the noise classification unit 15 may classify, as noise, a pulse detected at an interval shifted from an average pulse interval calculated from the pulse rate by a predetermined ratio (for example, ±10%) or more.

As described above, even if noise caused by the body motion or the like of the subject during the measurement of the blood pressure is erroneously detected as a pulse, the physiological information measurement device 100 according to the present embodiment can classify the noise to raise the possibility that the blood pressure can be calculated with a small number of measurement steps, which can reduce the burden on the subject.

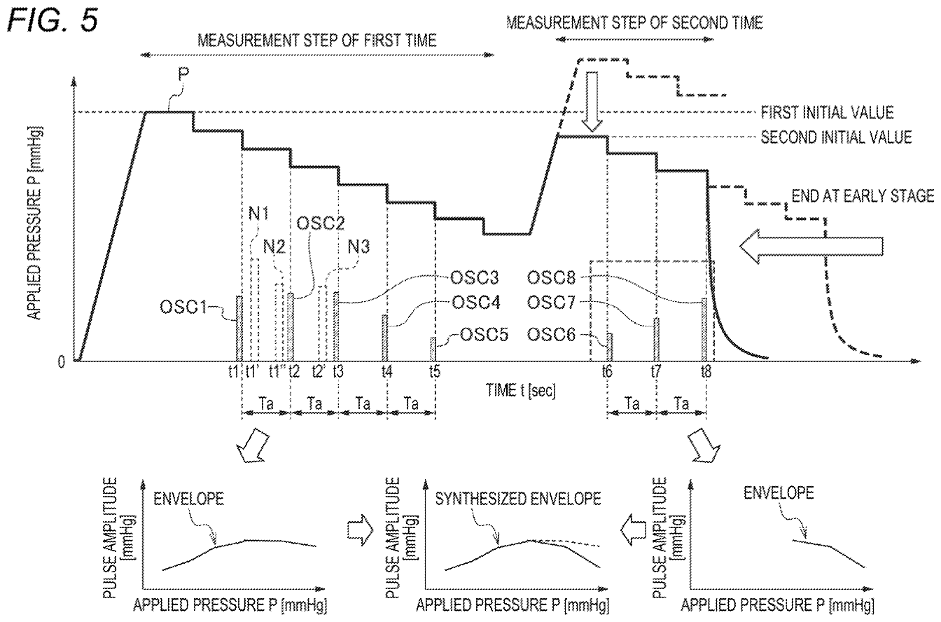

FIG. 5 is a graph illustrating another example of the pulse detection result of the physiological information measurement device according to the first embodiment of the presently disclosed subject matter. The horizontal axis represents the time t(s), and the vertical axis represents the applied pressure P (mmHg).

The example illustrated in FIG. 5 is a case where the peak value of the envelope cannot be identified from the plurality of pulses OSC1 to OSC5 classified as not noise in the measurement step of the first time compared to the example illustrated in FIG. 4.

In the measurement step of the first time, the noise classification unit 15 classifies the noises N1 to N3 as noise, and the noise classification unit 15 classifies the pulses OSC1 to OSC5 as not noise. From the amplitude of the pulses OSC1 to OSC5, the peak value of the envelope cannot be identified as illustrated in the lower left part of FIG. 5. Therefore, the mean arterial pressure (MAP) and the diastolic blood pressure (DIA) can be identified, but the systolic blood pressure (SYS) cannot be identified.

Accordingly, the blood pressure calculation possibility determination unit 16 determines that the blood pressure cannot be calculated from OSC1 to OSC5. At this time, the blood pressure calculation possibility determination unit 16 holds the pulses OSC1 to OSC5, which are classified as not noise.

In the measurement step of the second time, the pressure controller 11 sets a second initial value of the applied pressure P in the measurement step based on the determination result of the blood pressure calculation possibility determination unit 16 in the measurement step of the first time. At this time, the pressure controller 11 may set, as the second initial value, a minimum value within a range in which the pulse necessary for calculating the blood pressure can be detected. In the example of FIG. 5, in the measurement step of the second time, the minimum value within the range in which the pulses corresponding to the pulses OSC1 to OSC3 can be detected is set as the second initial value.

In the measurement step of the second time, the applied pressure P of the cuff 2 is stepwise deflated from the second initial value. In this period, the pulse detector 12 detects the pulses OSC6 to OSC8 at the time points t6, t7, and t8. In the example illustrated in FIG. 5, the pulses OSC6 to OSC8 are classified as not noise by the noise classification unit 15.

Next, the blood pressure calculation possibility determination unit 16 synthesizes the envelope calculated in the measurement step of the first time (lower left in FIG. 4) and the envelope calculated in the measurement step of the second time (lower left in FIG. 4), and calculates the synthesized envelope illustrated in the lower center of FIG. 4. Since the waveform of the synthesized envelope is convex upward, the peak value can be identified, and the systolic blood pressure (SYS) and the diastolic blood pressure (DIA) can be identified. Accordingly, the blood pressure calculation possibility determination unit 16 determines that the blood pressure can be calculated, and the blood pressure calculator 17 calculates the blood pressure from the synthesized envelope.

The time point t8 at which the pulse OSC8 is detected is in the middle of executing the deflation step in the measurement step of the second time. However, since the blood pressure can be calculated from the envelope synthesized at this time point, the pressure controller 11 cuts off the applied pressure P at the time point t8 and ends the measurement step of the second time at an early stage, as illustrated in FIG. 5.

As described above, even if the blood pressure cannot be calculated due to noise or the like using the envelope calculated in one measurement step, the physiological information measurement device 100 according to the present embodiment can synthesize the envelopes calculated in a plurality of measurement steps to raise the possibility that the blood pressure can be calculated. Accordingly, an unnecessary measurement step can be avoided, which can reduce the burden on the subject. If the pulse necessary for calculating the blood pressure is correctly detected and it is determined that the blood pressure can be calculated, the physiological information measurement device 100 according to the present embodiment can end the measurement step at an early stage, which can shorten the measurement time and reduce the burden on the subject.

### Second Embodiment

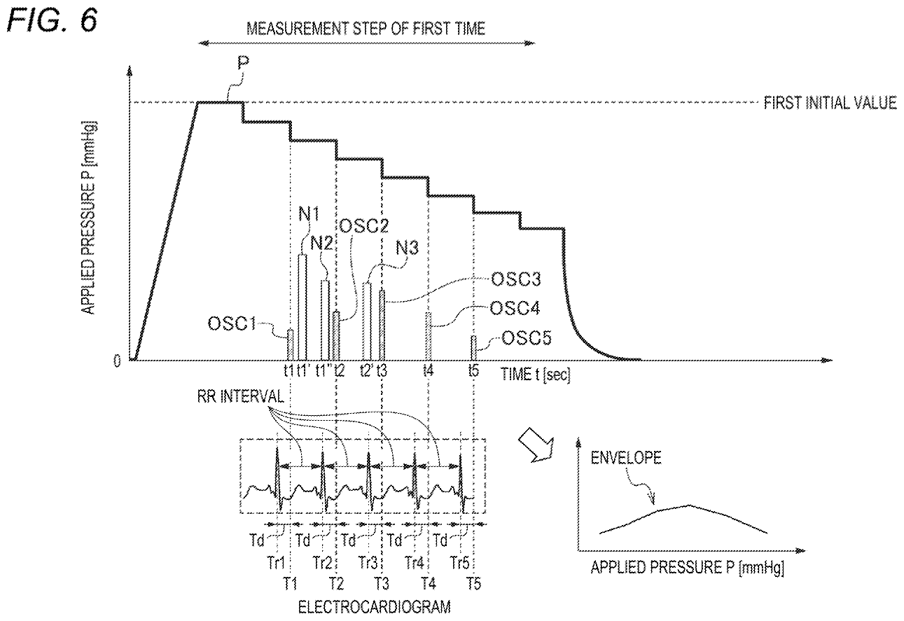

In the physiological information measurement device 100 according to the first embodiment described above, the physiological information is the heart rate, and the pulse detected at an interval deviated from the average beat interval Ta calculated from the heart rate by a predetermined ratio or more is classified as noise. On the other hand, in the physiological information device according to the second embodiment, the physiological information is an electrocardiogram, and a pulse detected at intervals shifted by a predetermined ratio (for example, ±10%) or more from an RR interval, which is an interval of peaks of the waveform in the electrocardiogram, is classified as noise. Alternatively, a pulse detected at a time point shifted by a predetermined ratio (for example, ±10%) or more with reference to time points TI to T5 obtained by adding a delay time Td to a time point Tr of the rising of the waveform in the electrocardiogram may be classified as noise. The reason for adding the delay time Td is that the detected pulse is delayed by the propagation time from the heart to the predetermined site where the cuff is wrapped, compared to the rising of the waveform in the electrocardiogram. The delay time Td may be the average propagation time of the pulses from the heart to the predetermined site where the cuff is wrapped. Hereinafter, a physiological information measurement device 200 according to the second embodiment will be described in detail with reference to FIG. 6.

FIG. 6 is a graph illustrating an example of the pulse detection result of the physiological information measurement device 200 according to the second embodiment of the presently disclosed subject matter. The horizontal axis represents the time t(s), and the vertical axis represents the applied pressure P (mmHg).

In the measurement step of the first time, the applied pressure P of the cuff 2 is stepwise deflated from the first initial value. During this period, the pulse detector 12 detects the pulse of the subject from the pressure oscillation detected by the pressure sensor 4. In the example illustrated in FIG. 6, the pulse detector 12 detects the pulses OSC1 to OSC5 at the time points t1, t2, t3, t4, and t5. Further, the pulse detector 12 detects the noises N1 to N3 as pulses at the time points t1′, t1″, and t2′. In the present embodiment, the noise classification unit 15 classifies whether the pulse detected by the pulse detector 12 is noise based on the physiological information detected by the physiological information detector 13. In the example illustrated in FIG. 6, the physiological information is an electrocardiogram, and a pulse detected at intervals shifted by a predetermined ratio (for example, #10%) or more from the RR interval in the electrocardiogram illustrated in the lower left part of FIG. 6 is classified as noise. Further, a pulse detected at the time point shifted by a predetermined ratio (for example, +10%) or more with reference to the time points TI to T5 obtained by adding the delay time Td to time points Tr1 to Tr5 of the rising of the waveform in the electrocardiogram is classified as noise. As a result, the noises N1 to N3 are classified as noise and the pulses OSC1 to OSC5 are classified as not noise. If the envelope is generated from the pulses OSC1 to OSC5, since the waveform of the envelope is convex upward as illustrated in the lower right part of FIG. 6, the peak value can be identified, and the systole and the diastole can be identified. Accordingly, the blood pressure calculation possibility determination unit 16 determines that the blood pressure can be calculated from the pulses OSC1 to OSC5, and the blood pressure calculator 17 calculates the blood pressure from the pulses OSC1 to OSC5.

As described above, even if noise caused by the body motion or the like of the subject during the measurement of the blood pressure is erroneously detected as a pulse, the physiological information measurement device 200 according to the present embodiment can classify the noise to raise the possibility that the blood pressure can be calculated, which can reduce the burden on the subject.

Although the embodiments of the presently disclosed subject matter have been described above, it is needless to say that the technical scope of the presently disclosed subject matter should not be construed as being limited to the description of the embodiments. The present embodiments are merely examples, and it is understood by those skilled in the art that various modifications of the embodiments are possible within the scope of the disclosed subject matters described in the claims. The technical scope of the presently disclosed subject matter should be determined based on the scope of the disclosed subject matters described in the claims and equivalents thereof.

Further, the processes of the physiological information measurement devices 100, 200 according to the embodiments can be implemented as a computer program that operates in the physiological information measurement devices 100, 200. That is, the physiological information measurement devices 100, 200, each can include a processor such as a CPU and a memory.

The program is stored in a non-transitory computer-readable medium and can be read by a computer. Examples of the non-transitory computer-readable medium include a magnetic recording medium, a magneto-optical recording medium, a CD-ROM, a CD-R, a CD-R/W, and a semi-conductor memory (including an EPROM and a flash ROM). Further, the program may be read by a computer using various types of temporary computer-readable media. Examples of the temporary computer-readable medium include an electric signal, an optical signal, and an electromagnetic wave. The temporary computer-readable medium can supply a program to the computer via a wired communication path such as an electric wire and an optical fiber or a wireless communication path.

What is claimed is:

1. A physiological information measurement device, comprising:
a pressure controller configured to control an applied pressure of a cuff to a predetermined site of a subject;
a pulse detector configured to detect pulses from a pressure received by the cuff from the predetermined site;
a physiological information detector configured to detect physiological information on the subject;
a noise classification unit configured to classify whether detection data detected by the pulse detector is noise based on the physiological information; and
a blood pressure calculator configured to calculate a blood pressure of the subject based on a plurality of the pulses that are classified as not noise by the noise classification unit.

2. The physiological information measurement device according to claim 1, wherein
the physiological information includes at least one of a heart rate, a pulse rate, or an electrocardiogram.

3. The physiological information measurement device according to claim 2, wherein
the noise classification unit classifies whether the detection data detected by the pulse detector is noise based on an average beat interval calculated from the heart rate, an average pulse interval calculated from the pulse rate, or an RR interval and a rising time point of a waveform in the electrocardiogram.

4. The physiological information measurement device according to claim 3, wherein
the noise classification unit calculates an interval of the detection data detected by the pulse detector, and classifies the detection data as noise if the detection data is detected at an interval deviated from the average beat interval or the average pulse interval by a predetermined ratio or more.

5. The physiological information measurement device according to claim 3, wherein
the noise classification unit
calculates an interval between peaks and the rising time point of the waveform of the detection data detected by the pulse detector, and
classifies the detected detection data as noise if the interval between peaks of the waveform of the detection data deviates from the RR interval by a predetermined ratio or more, or if the rising time point of the waveform of the detection data deviates from the rising time point of the waveform in the electrocardiogram by a predetermined ratio or more.

6. The physiological information measurement device according to claim 1, further comprising:
a blood pressure calculation possibility determination unit configured to determine whether the blood pressure can be calculated based on the pulse detected by the pulse detector, wherein
the pressure controller executes a measurement step of inflating or deflating the applied pressure from an initial value,
if the blood pressure calculation possibility determination unit determines that the blood pressure cannot be calculated in the measurement step of a first time, the pressure controller executes the measurement step of at least a second time, and
if the blood pressure calculation possibility determination unit determines that the blood pressure cannot be calculated in any measurement step, the pressure controller sets a minimum value within a range in which the pulse necessary for calculating the blood pressure can be detected as the initial value of the applied pressure in a next measurement step based on a determination result by the blood pressure calculation possibility determination unit.

7. A physiological information measurement method, comprising:
a pressure control step of controlling an applied pressure of a cuff to a predetermined site of a subject;
a pulse detection step of detecting pulses from a pressure received by the cuff from the predetermined site;
a physiological information detection step of detecting physiological information on the subject;
a noise classification step of classifying whether detection data detected in the pulse detection step is noise based on the physiological information; and
a blood pressure calculation step of calculating a blood pressure of the subject based on a plurality of the pulses that are classified as not noise in the noise classification step.

8. A non-transitory computer-readable storage medium storing a program for causing a computer to execute:
a pressure control step of controlling an applied pressure of a cuff to a predetermined site of a subject;
a pulse detection step of detecting pulses from a pressure received by the cuff from the predetermined site;
a physiological information detection step of detecting physiological information on the subject;
a noise classification step of classifying whether detection data detected in the pulse detection step is noise based on the physiological information, and classifying the detection data if the detection data is noise; and
a blood pressure calculation step of calculating a blood pressure of the subject based on a plurality of the pulses that are classified as not noise in the noise classification step.
